(19) United States
(12) Patent Application Publication
(10) Pub. No.: US 2025/0370797 A1
(43) Pub. Date: Dec. 4, 2025

(54) SYSTEMS AND METHODS FOR IDENTIFYING RESOURCE TRANSFERABILITY

(71) Applicant: Optum, Inc., Minnetonka, MN (US)

(72) Inventors: Divya SINGH, Hyderabad (IN); Pankaj SONI, Krishna Nagar (IN); Rishi SHARMA, Gurgaon (IN); Keerthika K, Bengaluru (IN)

(21) Appl. No.: 18/679,840

(22) Filed: May 31, 2024

(51) Int. Cl. G06F 9/50; G06F 11/30

(52) U.S. Cl. CPC G06F 9/5011; G06F 11/3051

(57) ABSTRACT

A model linking one or more environments with each of a plurality of conditions associated with the one or more environments is derived. A resource associated with a condition from the plurality of conditions requested by a user, and environment context data of the user are received. The model is applied to the environment context data of the user and the condition to identify a link of at least one environment, determined from the environment context data of the user, to the condition. Based on the identified link, a transferability of the resource requested is determined. Based on the determining, a status of the resource requested is changed, where the changing of the status initiates a transfer evaluation of the resource requested.

### TECHNICAL FIELD

The present disclosure relates generally to the field of data analytics, including artificial intelligence, and more particularly, to model-based systems and methods for identifying transferability of resources requested for environmentally induced conditions.

### BACKGROUND

Subrogation provides a payor the legal right to avoid or recover payments from a responsible party for various types of claims. For example, through subrogation, rights and/or duties of the payor are transferred to the responsible party. However, conventional systems and methods for identifying claims appropriate for subrogation (e.g., claims more likely to lead to a successful transfer of rights) are often limited to identifying only a subset of the types of claims that are subrogable. Resultantly, a significant proportion of claim costs remain improperly shifted away from the responsible party.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

The techniques of this disclosure improve the state of identification of transferable resources, and particularly with respect to resources associated with occupationally induced conditions.

In some aspects, the techniques described herein relate to a computer-implemented method. An example method includes: deriving, by one or more processors, a model linking one or more environments with each of a plurality of conditions associated with the one or more environments; receiving, by the one or more processors, a resource associated with a condition from the plurality of conditions requested by a user; receiving, by the one or more processors, environment context data of the user; applying, by the one or more processors, the model to the environment context data of the user and the condition to identify a link of at least one environment, determined from the environment context data of the user, to the condition; based on the identified link, determining, by the one or more processors, a transferability of the resource requested; and based on the determining, changing, by the one or more processors, a status of the resource requested, wherein the changing of the status initiates a transfer evaluation of the resource requested.

In other aspects, the techniques described herein relate to a system. An example system includes: one or more processors, and at least one memory storing instructions that, when executed by the one or more processors, cause the one or more processors to perform operations. The operations include: deriving a model linking one or more environments with each of a plurality of conditions associated with the one or more environments; receiving a resource associated with a condition from the plurality of conditions requested by a user; receiving environment context data of the user; applying the model to the environment context data of the user and the condition to identify a link of at least one environment, determined from the environment context data of the user, to the condition; based on the identified link, determining a transferability of the resource requested; and based on the determining, changing a status of the resource requested, wherein the changing of the status initiates a transfer evaluation of the resource requested.

In further aspects, the techniques described herein relate to a non-transitory computer readable medium. An example non-transitory computer readable medium stores instructions which, when executed by one or more processors, cause the one or more processors to perform operations. The operations include: deriving a model linking one or more environments with each of a plurality of conditions associated with the one or more environments; receiving a resource associated with a condition from the plurality of conditions requested by a user; receiving environment context data of the user; applying the model to the environment context data of the user and the condition to identify a link of at least one environment, determined from the environment context data of the user, to the condition; based on the identified link, determining a transferability of the resource requested; and based on the determining, changing a status of the resource requested, wherein the changing of the status initiates a transfer evaluation of the resource requested.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1 is a diagram showing an example of an environment for identifying resource transferability, according to some embodiments of the disclosure.

FIG. 2 is a flow chart showing an example process for identifying resource transferability, according to some embodiments of the disclosure.

FIG. 3 is a system flow diagram conceptually showing the process of FIG. 2 performed by one or more components of the environment of FIG. 1, according to some embodiments of the disclosure.

FIG. 4 depicts an example mapping table, according to some embodiments of the disclosure.

FIG. 5 is flow chart showing an example of a process for training, implementing, and monitoring a machine learning model, according to some embodiments of the disclosure.

FIG. 6 shows an implementation of a computer system that executes techniques presented herein, according to some embodiments of the disclosure.

### DETAILED DESCRIPTION

The present disclosure relates generally to the field of data analytics, including artificial intelligence, and more particularly, model-based systems and methods for identifying transferability of resources requested for environmentally induced conditions.

As briefly mentioned above, conventional systems and methods for identifying claims appropriate for subrogation (e.g., claims more likely to lead to a successful transfer of rights) are often limited to identifying only a subset of the types of claims that are subrogable. To provide an illustrative example, using conventional systems and methods, claims associated with medical and/or disability expenses related to occupational accidents or trauma are automatically identified and filtered for subrogation upon receipt by identifying codes indicative of occupational accidents included in the claims. Subrogation of these types of claims provides an insurance company the legal right to avoid or recover payments from a responsible party's insurance carrier (e.g., a workers' compensation carrier of an employer). However, conventional systems and methods are limited to only identifying claims related to occupational accidents or trauma for subrogation potential. There are no identification systems or methods for evaluating subrogation potential for claims related to occupational illnesses (e.g., diseases, disorders, or conditions associated with or inducible by workplace environment).

This is likely due to the higher guarantee of subrogation success of occupational accident claims based on the easier establishment of a direct line of liability from the employer to the employee facilitated by the timeliness of the occupational accident in relation to the filing of the claim. In contrast, many occupationally induced illnesses are chronic illnesses that develop or occur later in life as a result of an occupational exposure earlier in life. Because many of these chronic illnesses occur long after exposure has ended, the illnesses are generally not identified as workplace-related. Further, patients are often unaware that their condition could be due to an occupational exposure or is otherwise workplace-related, and therefore do not inform healthcare providers about their occupation. Resultantly, a number of subrogation cases reporting occupational illness is substantially low, and a significant proportion of occupational illness costs are improperly shifted from workers' compensation carriers to workers and their families, non-workers' compensation insurance carriers, and taxpayers.

The present disclosure solves this problem and/or other problems described above or elsewhere in the present disclosure, namely by describing an unconventional arrangement of a multi-component system and/or processes performed by the various components thereof that provide a specific and technical improvement over prior art systems. The specific and technical improvement includes the deriving and implementing of a model for identifying transferability of resources requested for environmentally induced conditions based on user environment context data, such as identifying a likelihood of subrogation of medical and/or pharmaceutical claims associated with occupationally induced conditions based on user occupational history.

Specifically, a model linking one or more environments with each of a plurality of conditions inducible by the one or more environments is derived. The model can be a mapping table or a trained machine learning model derived from comprehensive data sets for improved accuracy, the comprehensive data sets including known data for a plurality of resources associated with one or more of a plurality of conditions that have been previously requested by users having variable user environment context data, and evaluated for transferability. A user-requested resource associated with a condition from the plurality of conditions is received, along with environment context data of the user obtained from one or more external resources. The model is applied to the environment context data of the user and the condition to identify a link of at least one environment, determined from the environment context data of the user, to the condition. Based on the identified link, a transferability of the resource is determined, and a status of the resource requested is changed.

Changing of the status initiates a transfer evaluation of the resource requested. For example, a case is automatically opened and assigned in a separate evaluation system for the resource requested. Therefore, any time a new request for a resource is received, and without any user intervention involved, the request is processed as described above, and a new case is automatically opened and assigned if the processing identifies a transferability of the resource.

To help to increase an accuracy of the model, a feedback loop is generated to enable the model to be updated and/or adjusted based on feedback received from the evaluation system, where the feedback is related to a transfer outcome of the resource received. The transfer outcome indicates whether rights and/or duties associated with the resource were transferred to a responsible party, for example. Resultantly, the model is tuned, adjusted, or otherwise refined such that conditions, environments, and/or links identified between particular conditions and environments determined to have low success rates of transferability are reflected accordingly in the model such that resources associated with these conditions, environments, and/or particular links are filtered out and not assigned for transfer evaluation in future iterations of the process. Similarly, conditions, environments, and/or links identified between particular conditions and environments determined to have high success rates of transferability are reflected accordingly in the model such that resource requests received associated with these conditions, environments, and/or particular links are automatically flagged for transfer evaluation. Thus, an overall amount of data to be processed and analyzed is continuously reduced (e.g., saving computational resources), as prediction accuracy of the model is increased.

The technical improvements and advantages discussed above are not the sole improvements and advantages, and additional technical improvements and advantages will be discussed in the following sections. Further, based on the present disclosure, other technical improvements and advantages will be apparent to one of ordinary skill in the art.

Specific examples included throughout the present disclosure involve identifying transferability or subrogation potential of health insurance-related claims for occupational illness. However, it should be understood that techniques according to this disclosure are adaptable to other types of transferrable claims or resource requests, where environment history is a factor or variable (e.g., where the claims are for environmentally induced conditions). It should also be understood that the examples above and other examples presented in the present disclosure are illustrative only. The techniques and technologies of this disclosure are adaptable to any suitable activity.

Presented below are various aspects of machine learning techniques that can be adapted for processing data. As will be discussed in more detail below, the machine learning techniques include one or more aspects according to this disclosure, e.g., a particular selection of training data, a particular training process for a machine learning model, operation of the machine learning model in conjunction with particular data, modification of such particular data by the machine learning model, and/or other aspects that are apparent to one of ordinary skill in the art based on this disclosure.

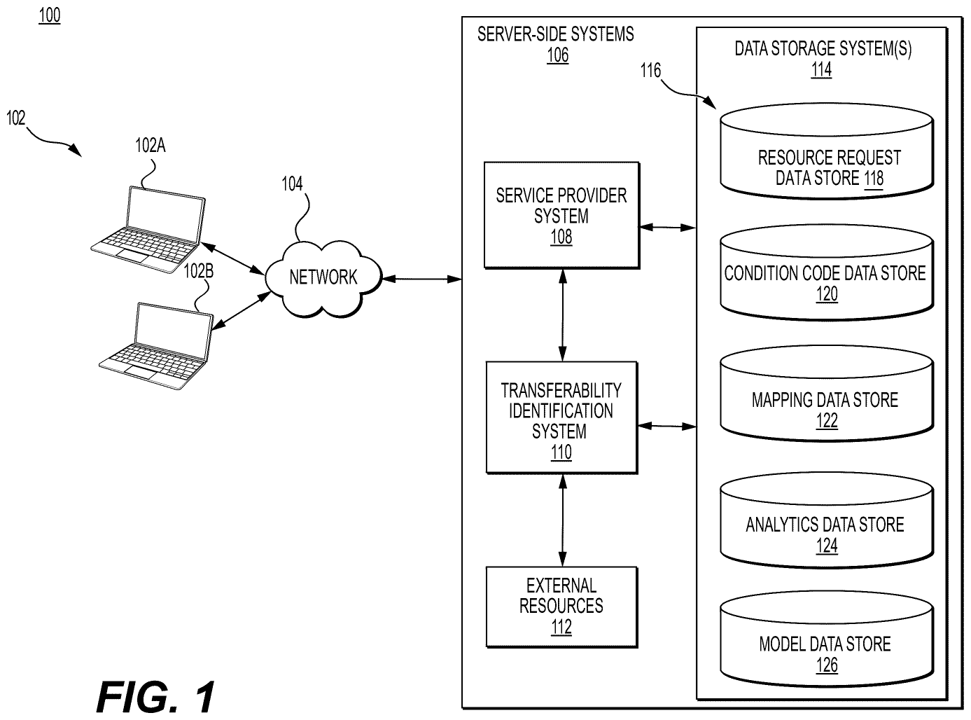

FIG. 1 is a diagram showing an example of an environment 100 for identifying resource transferability, according to some embodiments of the disclosure. A plurality of computing devices 102 associated with a plurality of users communicate with one or more other components of the environment 100 across a network 104, including one or more server-side systems 106. The server-side systems 106 include a service provider system 108, a transferability identification system 110, one or more external resources 112, and/or one or more data storage system(s) 114, among other systems.

In some examples, the service provider system 108, the transferability identification system 110, and/or the data storage system(s) 114 are associated with a common entity, e.g., a common payer or health plan provider, such as a health insurance company or the like offering private and/or public health care plans to individuals and/or families, among other health care-adjacent services. In such examples, the service provider system 108, the transferability identification system 110, and/or the data storage system(s) 114 can be part of a cloud service computer system (e.g., in a data center). That is, the various systems can be components or subsystems of a larger computer system.

In other examples, one or more of the service provider system 108, the transferability identification system 110, and/or the data storage system(s) 114 are separate systems associated with different entities. In such examples, each of the separate systems are communicatively connected to one another over the network 104 (e.g., via an application programming interface (API)). The systems and devices of the environment 100 can communicate in any arrangement. As will be discussed herein, systems and/or devices of the environment 100 communicate in order to perform target message generation.

The computing devices 102 are configured to enable the user to access and/or interact with other systems in the environment 100. In some examples, the computing devices 102 include a first subset of computing devices 102A and a second subset of computing devices 102B. The first subset of computing devices 102A are associated with healthcare providers, and are configured to provide or submit resource requests to the service provider system 108 over the network 104. The second subset of computing devices 102B are associated with members of a subrogation team for the service provider, and are configured to enable access to cases associated with a subset of the resource requests that are automatically opened and assigned based on determinations made by the transferability identification system 110.

Each of the computing devices 102 is a computer system such as, for example, a desktop computer, a laptop computer, a tablet, a smart cellular phone, a smart watch, or other wearable computer, etc. The computing devices 102 include one or more applications, e.g., a program, plugin, browser extension, etc., installed on a memory of the computing devices 102. The applications can include one or more of system control software, system monitoring software, software development tools, etc. In some embodiments, at least one of the applications is associated and configured to communicate with one or more of the other components in the environment 100, such as one or more of the server-side systems 106.

Additionally, one or more components of the computing devices 102, such as the at least one application, generate, or cause to be generated, one or more user interfaces based on instructions/information stored in the memory, instructions/information received from the other systems in the environment 100, and/or the like and cause the user interfaces to be displayed via a display of the computing devices 102. The user interfaces can be, e.g., mobile application interfaces or browser user interfaces and include text, input text boxes, selection controls, and/or the like. In some examples, the display includes a touch screen or a display with other input systems (e.g., a mouse, keyboard, etc.) to control the functions of the computing devices 102.

The service provider system 108 includes one or more server devices (or other similar computing devices) for executing services associated with a payer or health plan provider, such as an insurance company or other similar organization. One example service includes receiving and processing resource requests, such as medical and/or pharmaceutical claims, for a plurality of users or members having health plans provided by the payer, where claims data are stored in one of the data storage system(s) 114 described below. Another example service provided is a transferability identification service that can be provided by the payer or a third party to identify a subset of the resource requests having a potential (e.g., a likelihood or probability) of transfer or subrogation, as described in more detail with reference to the transferability identification system 110 below.

In some examples, the transferability identification system 110 is a system of (e.g., is hosted by) the same payer or health plan provider associated with the service provider system 108. In such examples, the transferability identification system 110 can be a sub-system or component of the service provider system 108. In other examples, the transferability identification system 110 is a system of (e.g., is hosted by) a third party that provides transferability identification services to the payer or health plan provider associated with the service provider system 108.

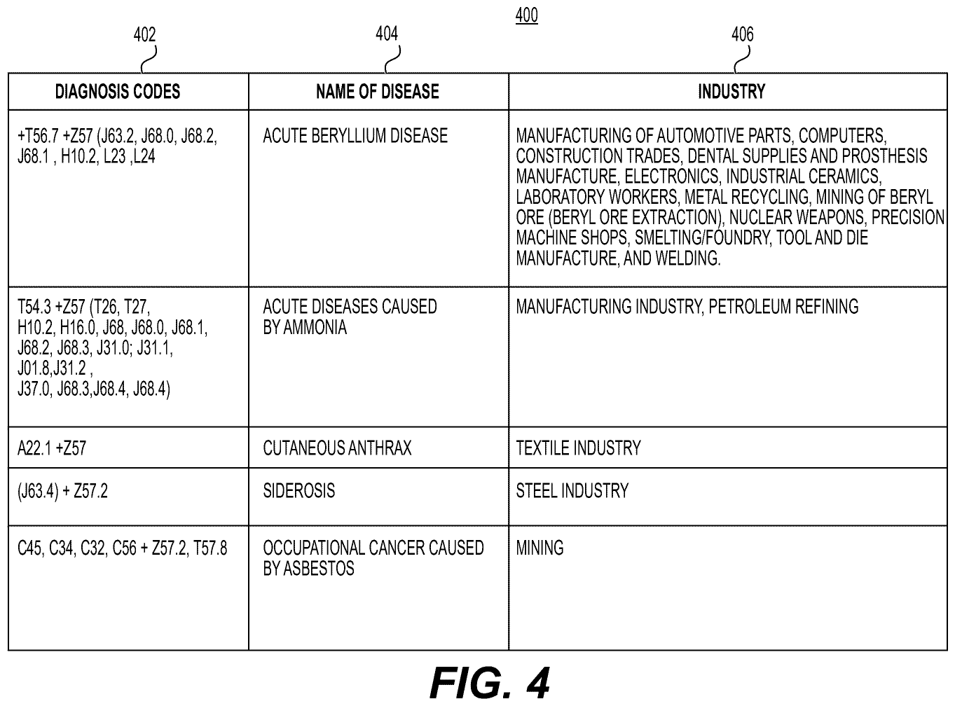

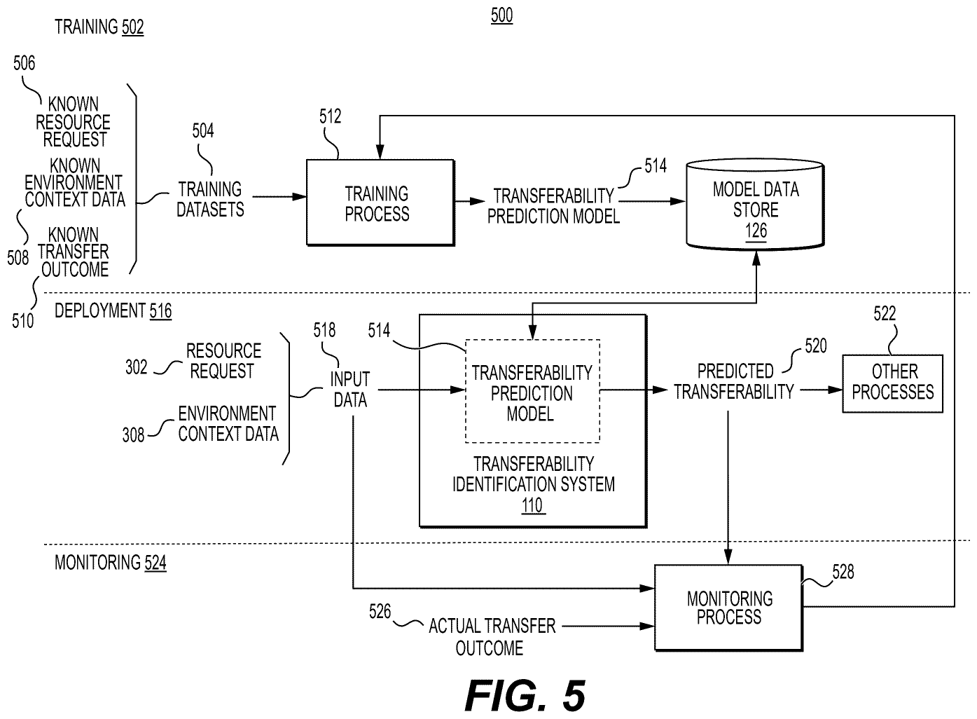

The transferability identification system 110 includes one or more server devices (or other similar computing devices) for performing operations related to transferability identification for resources, and particularly for resources associated with a plurality of conditions associated with or inducible by work-related environments (e.g., claims associated with occupational illnesses). The transferability identification system 110 derives and/or leverages a model to identify the transferability of these resources. In some examples, the model is a mapping table described in detail with reference to FIG. 4. In other examples, the model is a trained machine learning model described in detail with reference to FIG. 5.

The external resources 112 include various third party services configured to provide data utilized by the transferability identification system 110, as described in detail throughout the disclosure. Example external resources 112 include labor or employment-related services, directory services, public records searching services, and/or government-associated searching services, among other similar examples. To communicate with the external resources 112, the transferability identification system 110 generates API calls to the external resources 112 to request data, and receives the data from the external resources 112 responsive to the API calls.

The data storage system(s) 114 each include a server system or computer-readable memory such as a hard drive, flash drive, disk, etc. The data storage system(s) 114 include one or more data stores 116. The data stores 116 include and/or act as a repository or source for various types of data. Examples of the data stores 116 include a resource request data store 118, a condition code data store 120, a mapping data store 122, an analytics data store 124, and/or a model data store 126.

The resource request data store 118 includes, among other types of requested resources, a plurality of previously requested resources that are associated with one or more of the plurality of conditions inducible by work-related environments that have been received and/or processed by the service provider system 108 (e.g., historical resource requests or historical claims). For at least a subset of the historical resource requests that have undergone a transfer evaluation, the resource request data store 118 also includes known environment context data of a respective user that requested the resource and a known transfer outcome for the resource (e.g., whether or not the resource was able to be transferred or subrogated). In some examples, the data stored in the resource request data store 118 is used to derive the model leveraged by the transferability identification system 110.

The condition code data store 120 includes, for each condition of the plurality conditions, a listing of one or more condition codes associated with the condition. The conditions to be represented within the listing are identified and continuously updated using one or more of the external resources 112, such as an occupational disease listing periodically generated and published by the International Labour Organization (ILO). In some examples, the condition codes included within the listing for these conditions include international classification of diseases (ICD) codes for the conditions.

When the model leveraged by the transferability identification system 110 is a mapping table, the mapping data store 122 is configured to store the mapping table. The mapping table includes identified associations or links between condition codes and particular environments (e.g., workplace-related environments). In some examples, the condition code data store 120 and the mapping data store 122 comprise a single data store.

The analytics data store 124 includes analytics determined based on feedback and/or other similar historical data associated with transfer outcomes for resource requests having particular links (e.g., links of a particular condition to a particular one or more environments). The transfer outcome indicates whether transfer of the resource being requested was successful (e.g., the claim was subrogated) or not when the particular link was present. In some instances, transferability of a resource associated with a same condition can be variable among different environments linked to the condition, because certain environments have less evidence of causation or inducibility of the condition than others environments, which can impact the ability to transfer resources associated with the condition when only links to the certain environments are present. In some examples, the analytics data store 124 store ratios or percentage of resource requests that have been successful for particular links that can be compared to a threshold, as described below. In other examples, the analytics data store 124 includes a listing of links that meet the threshold, and/or a listing of links that do not meet the threshold for reference by the transferability identification system 110.

When the model leverage by the transferability identification system 110 is a trained machine learning model, the model data store 126 stores the trained machine learning model for subsequent retrieval and execution by the transferability identification system 110.

In some examples, one of the data storage system(s) 114 maintains each of the data stores 116. In other examples, one or more of the data stores 116 are maintained across two or more different ones of the data storage system(s) 114. One or more of the data storage system(s) 114 can be a system of (e.g., hosted by) the same payer or health plan provider associated with the service provider system 108 and/or transferability identification system 110. Additionally or alternatively, one or more of the data storage system(s) 114 are associated with a third party that provides data storage services to the service provider system 108 and/or transferability identification system 110.

The network 104 over which the one or more components of the environment 100 communicate includes one or more wired and/or wireless networks, such as a wide area network (“WAN”), a local area network (“LAN”), personal area network (“PAN”), a cellular network (e.g., a 3G network, a 4G network, a 5G network, etc.) or the like. In some embodiments, the network 104 includes the Internet, and information and data provided between various systems occurs online. “Online” means connecting to or accessing source data or information from a location remote from other devices or networks coupled to the Internet. Alternatively, “online” refers to connecting or accessing a network (wired or wireless) via a mobile communications network or device. The Internet is a worldwide system of computer networks-a network of networks in which a party at one computer or other device connected to the network can obtain information from any other computer and communicate with parties of other computers or devices. The computing devices 102 and one or more of the server-side systems 106 are connected via the network 104, using one or more standard communication protocols. The computing devices 102 and the one or more of the server-side systems 106 transmit and receive communications from each other across the network 104.

Although depicted as separate components in FIG. 1, it should be understood that a component or portion of a component in the system of the environment 100 is, in some embodiments, integrated with or incorporated into one or more other components. As one example, the transferability identification system 110 and/or one or more of the data storage system(s) 114 can be integrated with the service provider system 108 or the like. In some embodiments, operations or aspects of one or more of the components discussed above are distributed amongst one or more other components. Any suitable arrangement and/or integration of the various systems and devices of the environment 100 can be used.

In the following disclosure, various acts are described as performed or executed by a component from FIG. 1, such as the computing devices 102 or one or more of the server-side systems 106, or components thereof. However, it should be understood that in various aspects, various components of the environment 100 discussed above execute instructions or perform acts including the acts discussed below. An act performed by a device is considered to be performed by a processor, actuator, or the like associated with that device. Further, it should be understood that in various embodiments, various steps can be added, omitted, and/or rearranged in any suitable manner.

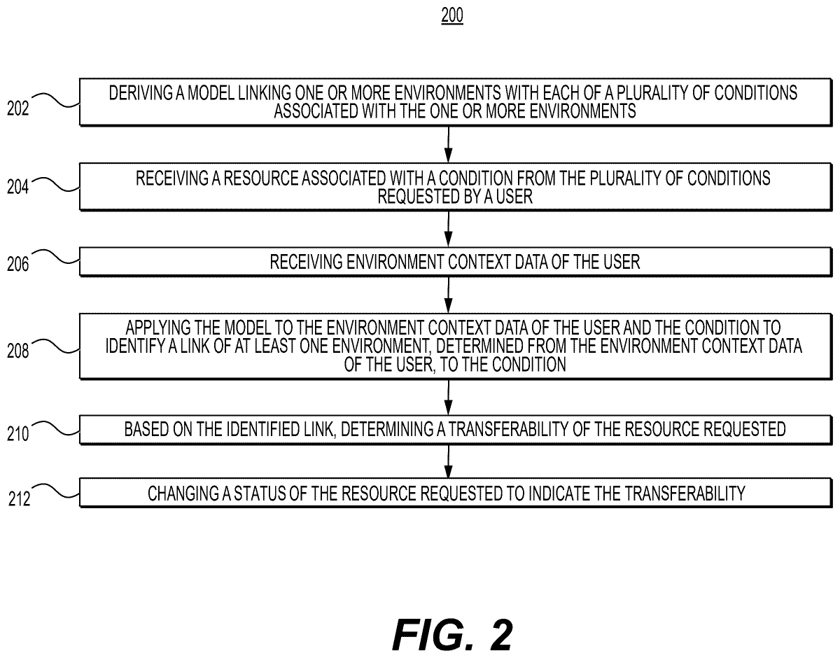

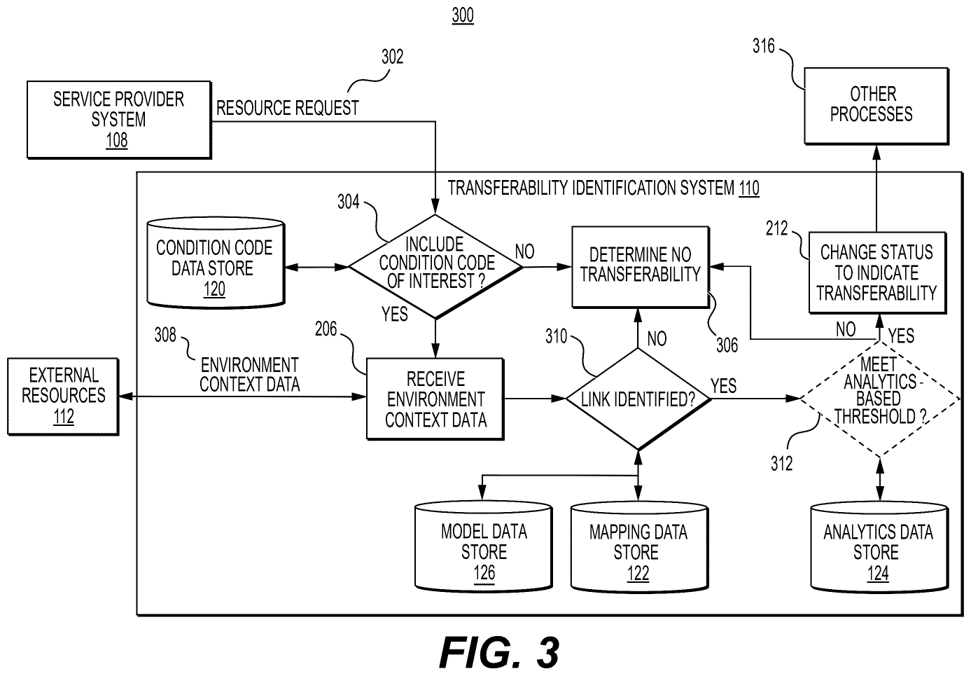

FIG. 2 is a flow chart showing an example method 200 for identifying resource transferability, and FIG. 3 is a system flow diagram 300 depicting the method 200 of FIG. 2, according to some embodiments of the disclosure. In some examples, the method 200 is performed by the transferability identification system 110.

Referring concurrently to FIGS. 2 and 3, at step 202, the method 200 includes deriving a model linking one or more environments with each of a plurality of conditions associated with the one or more environments. Example environments include occupational industries (e.g., manufacturing industry, textile industry, steel industry, mining, construction trade, etc.). Example conditions include types of conditions that can be inducible (e.g., caused, at least in part) by typical working environments present in these occupational industries. In other words, the conditions are occupational diseases, illnesses, disorders, or conditions.

In some examples, and as described in more detail below with reference to FIG. 4, the model is a mapping table that is generated and stored in the mapping data store 122 to enable subsequent querying. In other examples, and as described in more detail below with reference to FIG. 5, the model derived is a machine learning model that is generated and stored in the model data store 126 to enable subsequent retrieval and execution.

At step 204, the method 200 includes receiving a resource associated with a condition from the plurality of conditions requested by a user. For example, the resource is received as a resource request 302 from the service provider system 108. The condition that the resource is associated with is indicated by a code included in the resource request 302. To provide an illustrative example, the resource request 302 can be a claim (e.g., a medical and/or pharmaceutical claim) that includes an ICD code indicative of the condition. As one illustrative example, the claim includes the ICD code G-92 indicative of toxic encephalopathy. The resource is described herein as being associated with one condition for clarity and brevity. However, in other examples, a resource can be associated with more than one condition from the plurality of conditions.

In some examples and as shown in FIG. 3, upon receipt of the resource request 302, a determination as to whether the resource request 302 includes a condition code of interest (e.g., whether the code included in the resource request 302 is a code of interest) is made at decision 304. For example, using the code, the transferability identification system 110 queries the condition code data store 120 to determine whether the code included in the resource request 302 corresponds to a code within the listing of codes of interest stored by the condition code data store 120. The codes of interest within the listing are codes indicating conditions that are inducible by (e.g., can be caused, at least in part, by) workplace or occupational environment. In other words, the codes of interest within the listing are codes indicating conditions classified as occupational diseases, illnesses, disorders, or conditions.

If at the decision 304, a determination is made that the resource request 302 does not include any condition code of interest, then the transferability identification system 110 determines no transferability of the resource at step 306, and the method 200 ends. Otherwise, if at the decision 304, a determination is made that the resource request 302 includes at least one condition code of interest, then the method proceeds to step 206.

At step 206, the method 200 includes receiving environment context data 308 of the user. The environment context data 308 includes at least an occupational history of the user. For example, user information, such as name and date of birth, is extracted from the resource request 302. At least a portion of the user information can be provided to one of the external resources 112, such as a directory service (e.g., whitepages.com), to identify the user and obtain further user information, such as family members, address, phone number, and/or current occupation, to facilitate a subsequent search for occupational history of the user. The user information obtained from the resource request 302 and/or the directory service is then provided to another one of the external resources 112, such as a public records searching service (e.g., truthfinder.com) to identify the occupational history of the user. Alternatively, an identifier of the user included in the resource request 302 such as a social security number of the user, can be used to obtain the occupational history of the user through a government-provided service (e.g., one of the external resources 112), for example.

For each of one or more entities included in the user's occupational history (e.g., past and/or present employers), one or more associated industries can be identified as one or more environments that the user has been exposed to. For example, the entities are provided to one of the external resources 112, such as a business information and industry classification service (e.g., siccode.com), to obtain the associated industries, and thus environments. To provide an illustrative example, an occupational history of the user indicates an industrial adhesives manufacturer as a past employer, and the industrial adhesives manufacturer is identified by the business information and industry classification service as being associated with the automotive industry.

To send requests to or otherwise communicate with any of the above-described external resources 112 to obtain the environment context data 308, the transferability identification system 110 generates API calls to the external resources 112 that include any necessary identifying data to fulfill the requests, and receives various portions of the environment context data 308 of the user from the external resources 112 responsive to the API calls.

At step 208, the method 200 includes applying the model to the environment context data 308 of the user and the condition to identify a link of at least one environment, determined from the environment context data 308 of the user, to the condition. The link indicates or suggests that the at least one environment to which the user has been exposed to (e.g., determined based on the user's occupational history) is prone to inducing this type of condition.

In some examples, and as described in more detail with reference to FIG. 4 below, when the model is the mapping table, the transferability identification system 110 queries the mapping table stored in the mapping data store 122 using the at least one environment and the condition to determine whether there is an association or mapping, and thus a link therebetween, indicated by the mapping table. The query result (e.g., output) indicates a link or a lack thereof. Continuing the previous example, the automotive industry and the toxic encephalopathy condition are used to query the mapping table to determine whether the mapping table indicates or maps toxic encephalopathy as being a type of condition inducible by the automotive manufacturing industry.

In some examples, when the model is the trained machine learning model, the transferability identification system 110 retrieves and executes the trained machine learning model from the model data store 126. For example, and as described with reference to FIG. 5 in more detail, the resource request 302 and the environment context data 308 are provided as input to the trained machine learning model for processing. The trained machine learning then provides, as output, a predicted transferability (e.g., a probability or likelihood that the resource can be transferred or subrogated). The predicted transferability is indicative of a link or a lack thereof. For example, a predictive transferability above a predefined threshold is indicative of the link.

As part of the step 208 and as shown in FIG. 3, based on the output of the model, a determination as to whether a link is identified is made at decision 310. If at the decision 310, a determination is made that no link is identified, then the transferability identification system 110 determines no transferability of the resource at step 306, and the method 200 ends. Otherwise, if at the decision 310, a determination is made that at least one link is identified, the method 200 proceeds to step 210.

Optionally, prior to proceeding to the step 210, a determination as to whether the link identified meets an analytics-based threshold is made at optional decision 312. For example, the analytics data store 124 is queried to determine whether, based on historical transfer analytics, the success rate of attempted transfers of resources including the link identified (e.g., the particular condition and the particular at least one environment) meets a threshold success rate. The success rate, in some instances, can be variable because certain environments have less evidence of causation or inducibility of a given condition than others, which impacts the ability to transfer resources associated with the given condition when only links to the certain environments are present. To provide an illustrative example, a ratio or percentage of claims associated with toxic encephalopathy that were attempted and successfully subrogated for users having worked in the automotive industry is obtained and compared against the threshold. Alternatively, the analytics data store 124 includes a listing of links that meet the threshold, and/or a listing of links that do not meet the threshold that are referenced at the optional decision 312. If at the optional decision 312, a determination is made that the link identified does not meet the analytics-based threshold, then the transferability identification system 110 determines no transferability of the resource at step 306, and the method 200 ends. Otherwise, if at the optional decision 312, a determination is made that the link identified meets the analytics-based threshold, the method 200 proceeds to step 210.

At step 210, the method 200 includes, based on the identified link, determining a transferability of the resource requested. At step 212, the method 200 includes, based on the determining, changing a status of the resource requested to indicate the transferability thereof. For example, the resource request 302 is flagged, marked, or otherwise altered to differentiate the resource request 302 from other resource requests that were determined to have no transferability by the transferability identification system 110.

In some examples, a summary including the condition, the at least one environment, and/or the identified link is generated and stored as part of (e.g., is used to alter the resource request 302) or is stored in association with the resource request 302.

In further examples, the changing of the status initiates a transfer evaluation of the resource requested (e.g., one of other processes 316 shown in FIG. 3). Initiation of the transfer evaluation can include automatically opening and assigning a case associated with the resource requested. The summary can be stored in association with the case. The case is opened and assigned as part of a resource transfer or subrogation service offered by the service provider or a third party. For example, a subrogation team member to which the case is assigned is able to view and/or select the case upon launching an application associated with the subrogation service on one of the second subset of computing devices 102B to enable investigation of the case. In some examples, when the model is the trained machine learning model configured to output a predicted transferability, a value of the predicted transferability is used to rank, order, prioritize, or otherwise enable triage of the case among a plurality of other assigned cases.

Once the transfer evaluation of the resource requested has been completed, an outcome of the transfer evaluation (e.g., a transfer outcome or transfer outcome data) is received by the transferability identification system 110 as feedback. The model derived at step 202 and applied at step 208 is updated based on the feedback to improve an accuracy of the model, as described in more detail with reference to FIGS. 4 and 5 below. Additionally, the feedback can be further used to eliminate one or more codes from the plurality of codes stored in the condition code data store 120. For example, if throughout the course of the transfer evaluation, a particular condition is identified as being an unlikely condition for which a resource transfer will be requested (e.g., because evidence of causation of the particular condition by a particular environment is limited), a condition code indicating that particular condition is removed from the listing stored in the condition code data store 120. Resultantly, any future resource requests received that are associated with the particular condition are not filtered for further analysis (e.g., the requests are determined to have no transferability at step 306 based on the determination made at decision 304).

Accordingly, certain aspects of this disclosure include methods for identifying resource transferability. The method 200 and corresponding system flow diagram 300 described above are provided merely as examples, and can include additional, fewer, different, or differently arranged steps than depicted in FIGS. 2 and 3.

FIG. 4 depicts an example mapping table 400, according to some embodiments of the disclosure. The mapping table 400 is one example type of model derived to link each of a plurality environments with one or more of a plurality of conditions associated with (e.g., inducible by) the respective environments.

As shown in FIG. 4, the mapping table 400 includes a plurality of columns 402, 404, 406. A first column 402 corresponds to codes indicative of conditions (e.g., “Diagnosis Codes”). A second column 404 corresponds to a common name for type(s) of conditions (e.g., “Disease Name”). A third column 406 corresponds to environments that can induce the conditions (e.g., “Industry”). Therefore, each row of the mapping table 400 represents a particular type of condition and depicts, for that type of condition, any codes indicative of that condition in the first column 402, the common name of that condition in the second column 404, and any environments that can induce or cause that condition in the third column 406.

To apply the mapping table 400 (e.g., at step 208 of the method 200), the condition associated with the resource request 302, and specifically the condition code indicative of the condition included in the resource request 302, is used to query the mapping table 400 (e.g., is used as a look-up value) to identify at least one row including the condition code within the first column 402. Then, a link is determined when the environments listed in the third column 406 for that row include at least one environment that is identified from the environment context data 308 of the user.

The mapping table 400 is generated or derived based on known data for a plurality of resources requested by a plurality of users (e.g., known data for historical resource requests stored in resource request data store 118). The known data for each of the resources includes one or more of the plurality of conditions associated with the respective resource, environment context data of the respective user (e.g., at a time of the resource was requested), and a transfer outcome of the respective resource.

In some examples, the mapping table 400 is updated or adjusted. For example, transfer outcomes of a subset of resources requested, for which transferability thereof was identified by the transferability identification system 110, are received as feedback from the transfer evaluation. The feedback is analyzed and/or stored, for example, in the analytics data store 124. The mapping table 400 is then updated based on the feedback. As one example, certain environments have less evidence of causation or inducibility of a given condition than others. If based on analytics, a determination is made that a resource (e.g., medical and/or pharmaceutical claim costs) associated with a particular condition is highly unlikely to be transferred when a link is only present for those certain environments having less evidence of causation or inducibility, such environments are removed from the third column 406 for the row representing the particular condition.

FIG. 5 is flow chart 500 showing an example of a process for training and implementing a machine learning model, according to some embodiments of the disclosure. In some embodiments, the transferability identification system 110 one or more of generates, stores, trains, or uses a machine learning model, such as a transferability prediction model 514, configured to predict a transferability of a resource requested by a user based on a condition associated with the resource and environment context data of the user. The transferability identification system 110 includes a machine learning model and/or instructions associated with the machine learning model, e.g., instructions for generating a machine learning model, training the machine learning model, using the machine learning model, etc. In other embodiments, a system or device other than the transferability identification system 110 is used to generate and/or train the machine learning model. For example, such a system includes instructions for generating the machine learning model and the training data, and/or instructions for training the machine learning model. A resulting trained-machine learning model is then provided to the transferability identification system 110 for use.

As depicted in FIG. 5, in some examples, the process includes a training phase 502, a deployment phase 516, and a monitoring phase 524. In the training phase 502, at step 512, the process includes receiving and processing training datasets 504 to generate (e.g., build) a trained transferability prediction model 514 for predicting a transferability of a resource requested by a user.

The training datasets 504 are associated with a plurality of users who have previously requested resources (e.g., previously submitted claims) that are associated with one or more of the plurality of conditions inducible by work-related environments and have been evaluated for transferability. In some examples, a given training dataset 504 includes a known resource associated with one or more of the plurality of conditions requested by a respective user (e.g., a known resource request 506), a known environment context data 508 of the respective user at a time of the transfer evaluation of the known resource, and/or a known transfer outcome 510 of the known resource. The training datasets 504 are generated, received, or otherwise obtained from internal and/or external resources. For example, the training datasets 504 include portions of data collected by the service provider system 108 and stored in the resource request data store 118 and/or portions of data obtained from the external resources 112. Additionally, or alternatively, the training datasets 504 include datasets obtained from a third party and/or a public database.

Generally, a model includes a set of variables, e.g., nodes, neurons, filters, etc., that are tuned, e.g., weighted or biased, to different values via the application of the training datasets 504. In some examples, the training process at step 512 employs supervised, unsupervised, semi-supervised, and/or reinforcement learning processes to train the model (e.g., to result in the trained transferability prediction model 514). In some embodiments, a portion of the training datasets 504 are withheld during training and/or used to validate the trained transferability prediction model 514.

When supervised learning processes are employed, labels or scores facilitate the learning process by providing a ground truth. For example, the labels or scores indicate the known transfer outcome 510 of the known resource (e.g., whether the resource was able to be transferred or subrogated). Training proceeds by feeding the known resource request 506 and the known environment context data 508, from one of the plurality of training datasets 504, into the model, the model having variables set at initialized values, e.g., at random, based on Gaussian noise, a pre-trained model, or the like. The model outputs a predicted transferability. The output is compared with the corresponding label or score (e.g., the ground truth), and in this case the known transfer outcome 510, to determine an error, which is then back-propagated through the model to adjust the values of the variables. This process is repeated for a plurality of the training datasets 504 at least until a determined loss or error is below a predefined threshold. In some examples, some of the training datasets 504 are withheld and used to further validate or test the trained transferability prediction model 514.

For unsupervised learning processes, the training datasets 504 do not include pre-assigned labels or scores to aid the learning process (e.g., do not include the known transfer outcome 510). Rather, unsupervised learning processes include clustering, classification, or the like to identify naturally occurring patterns in the training datasets 504. Supervised or unsupervised K-means clustering or K-Nearest Neighbors can also be used. Combinations of K-Nearest Neighbors and an unsupervised cluster technique can also be used. For semi-supervised learning, a combination of the training datasets 504 with pre-assigned labels or scores and the training datasets 504 without pre-assigned labels or scores are used to train the model.

When reinforcement learning is employed, an agent (e.g., an algorithm) is trained to make a decision regarding the transferability of resources from the training datasets 504 through trial and error. For example, upon making a decision, the agent then receives feedback (e.g., a positive reward if the predicted transferability for a resource aligns with an actual transfer outcome for the resource), adjust its next decision to maximize the reward, and repeat until a loss function is optimized.

Once trained, the trained transferability prediction model 514 is stored (e.g., in the model data store 126) and subsequently executed by one of the server-side systems 106, such as the transferability identification system 110, during the deployment phase 516. For example, during the deployment phase 516, the trained transferability prediction model 514 receives input data 518, including the resource request 302 and the environment context data 308, as described above in detail with reference to FIGS. 2 and 3. The trained transferability prediction model 514 outputs a predicted transferability 520 associated with the resource request 302. The predicted transferability 520 is provided as further input into one or more other processes 522. For example, and as discussed in detail with reference to FIG. 3, the predicted transferability 520 is optionally compared to an analytics-based threshold, a status associated with the resource request 302 is changed to indicate transferability, and/or a transfer evaluation of the resource request 302 is initiated, among other of the processes 316.

During the monitoring phase 524, an actual transfer outcome 526 is collected and received as feedback. During process 528, the actual transfer outcome 526 is analyzed along with the input data 518 to determine an accuracy of the predicted transferability 520. In some examples, based on the analysis, the process returns to the training phase 502, where at step 512 values of one or more variables of the model are adjusted or tuned.

The process described above is provided merely as an example, and can include additional, fewer, different, or differently arranged aspects than depicted in FIG. 5.

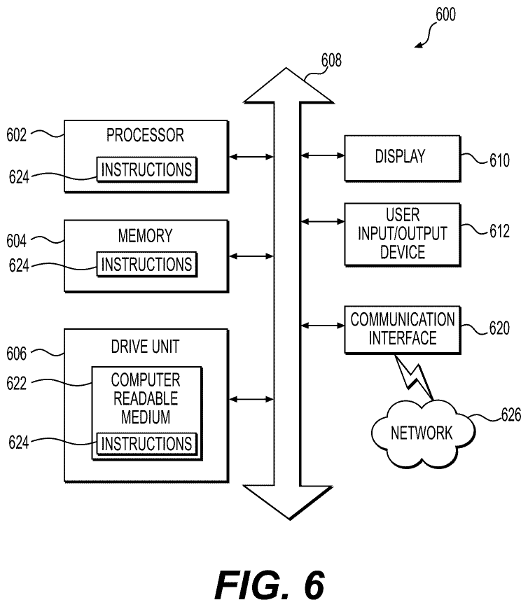

FIG. 6 shows an implementation of a computer system 600 that executes techniques presented herein, according to some embodiments of the disclosure. The computer system 600 can include a set of instructions that can be executed to cause the computer system 600 to perform any one or more of the methods or computer-based functions disclosed herein. The computer system 600 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 600 operates in the capacity of a server or as a client user computer in a server-client user network environment, or as a peer computer system in a peer-to-peer (or distributed) network environment. The computer system 600 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 600 can be implemented using electronic devices that provide voice, video, or data communication. Further, while the computer system 600 is illustrated as a single system, the term “system” shall also be taken to include any collection of systems or sub-systems that individually or jointly execute a set, or multiple sets, of instructions to perform one or more computer functions.

As illustrated in FIG. 6, the computer system 600 includes a processor 602, e.g., a central processing unit (CPU), a graphics processing unit (GPU), or both. The processor 602 can be a component in a variety of systems. For example, the processor 602 is part of a standard personal computer or a workstation. The processor 602 is one or more processors, digital signal processors, application specific integrated circuits, field programmable gate arrays, servers, networks, digital circuits, analog circuits, combinations thereof, or other now known or later developed devices for analyzing and processing data. The processor 602 implements a software program, such as code generated manually (e.g., programmed).

The computer system 600 includes a memory 604 that can communicate via a bus 608. The memory 604 is a main memory, a static memory, or a dynamic memory. The memory 604 includes, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media, and the like. In one implementation, the memory 604 includes a cache or random-access memory for the processor 602. In alternative implementations, the memory 604 is separate from the processor 602, such as a cache memory of a processor, the system memory, or other memory. The memory 604 can be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 604 is operable to store instructions executable by the processor 602. The functions, acts or tasks illustrated in the figures or described herein are performed by the processor 602 executing the instructions stored in the memory 604. The functions, acts or tasks are independent of the particular type of instructions set, storage media, processor or processing strategy and are performed by software, hardware, integrated circuits, firm-ware, micro-code and the like, operating alone or in combination. Likewise, processing strategies can include multiprocessing, multitasking, parallel processing, and the like.

As shown, the computer system 600 further included a display 610, such as a liquid crystal display (LCD), an organic light emitting diode (OLED), a flat panel display, a solid-state display, a cathode ray tube (CRT), a projector, a printer or other now known or later developed display device for outputting determined information. The display 610 acts as an interface for the user to see the functioning of the processor 602, or specifically as an interface with the software stored in the memory 604 or in a drive unit 606.

Additionally or alternatively, the computer system 600 includes an input/output device 612 configured to allow a user to interact with any of the components of the computer system 600. The input/output device 612 is a number pad, a keyboard, or a cursor control device, such as a mouse, or a joystick, touch screen display, remote control, or any other device operative to interact with the computer system 600.

The computer system 600 also or alternatively includes the drive unit 606 implemented as a disk or optical drive. The drive unit 606 includes a computer-readable medium 622 in which one or more sets of instructions 624, e.g., software, can be embedded. Further, the sets of instructions 624 embody one or more of the methods or logic as described herein. The instructions 624 reside completely or partially within the memory 604 and/or within the processor 602 during execution by the computer system 600. The memory 604 and the processor 602 can also include computer-readable media as discussed above.

In some systems, the computer-readable medium 622 includes the sets of instructions 624 or receives and executes the sets of instructions 624 responsive to a propagated signal so that a device connected to a network 626 can communicate voice, video, audio, images, or any other data over the network 626. Further, the sets of instructions 624 are transmitted or received over the network 626 via a communication port or interface 620, and/or using the bus 608. The communication port or interface 620 is a part of the processor 602 or is a separate component. The communication port or interface 620 is created in software or is a physical connection in hardware. The communication port or interface 620 are configured to connect with the network 626, external media, the display 610, or any other components in the computer system 600, or combinations thereof. The connection with the network 626 is a physical connection, such as a wired Ethernet connection or is established wirelessly as discussed below. Likewise, the additional connections with other components of the computer system 600 are physical connections or are established wirelessly. The network 626 is alternatively directly connected to the bus 608.

While the computer-readable medium 622 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that cause a computer system to perform any one or more of the methods or operations disclosed herein. In some examples, the computer-readable medium 622 is non-transitory, and is tangible.

The computer-readable medium 622 can include a solid-state memory such as a memory card or other package that houses one or more non-volatile read-only memories. The computer-readable medium 622 can be a random-access memory or other volatile re-writable memory. Additionally or alternatively, the computer-readable medium 622 can include a magneto-optical or optical medium, such as a disk or tapes or other storage device to capture carrier wave signals such as a signal communicated over a transmission medium. A digital file attachment to an e-mail or other self-contained information archive or set of archives are considered a distribution medium that is a tangible storage medium. Accordingly, the disclosure is considered to include any one or more of a computer-readable medium or a distribution medium and other equivalents and successor media, in which data or instructions are storable.

In an alternative implementation, dedicated hardware implementations, such as application specific integrated circuits, programmable logic arrays and other hardware devices, can be constructed to implement one or more of the methods described herein. Applications that include the apparatus and systems of various implementations can broadly include a variety of electronic and computer systems. One or more implementations described herein implement functions using two or more specific interconnected hardware modules or devices with related control and data signals that can be communicated between and through the modules, or as portions of an application-specific integrated circuit. Accordingly, the present system encompasses software, firmware, and hardware implementations.

The computer system 600 is connected to the network 626. The network 626 defines one or more networks including wired or wireless networks, such as the network 104 described in FIG. 1. The wireless network can be a cellular telephone network, an 602.11, 602.16, 602.20, or WiMAX network. Further, such networks include a public network, such as the Internet, a private network, such as an intranet, or combinations thereof, and utilize a variety of networking protocols now available or later developed including, but not limited to TCP/IP based networking protocols. The network 626 can include wide area networks (WAN), such as the Internet, local area networks (LAN), campus area networks, metropolitan area networks, a direct connection such as through a Universal Serial Bus (USB) port, or any other networks that allow for data communication. The network 626 is configured to couple one computing device to another computing device to enable communication of data between the devices. The network 626 generally is enabled to employ any form of machine-readable media for communicating information from one device to another. The network 626 includes communication methods by which information travels between computing devices. The network 626 can be divided into sub-networks. The sub-networks allow access to all of the other components connected thereto or the sub-networks restrict access between the components. The network 626 can be regarded as a public or private network connection and can include, for example, a virtual private network or an encryption or other security mechanism employed over the public Internet, or the like.

In accordance with various implementations of the present disclosure, the methods described herein are implemented by software programs executable by a computer system. Further, in one example, non-limited implementation, implementations can include distributed processing, component/object distributed processing, and parallel processing. Alternatively, virtual computer system processing can be constructed to implement one or more of the methods or functionalities as described herein.

Although the present specification describes components and functions that are implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (e.g., computer) system executing instructions (computer-readable code) stored in storage. It will also be understood that the disclosure is not limited to any particular implementation or programming technique and that the disclosure is implementable using any appropriate techniques for implementing the functionality described herein. The disclosure is not limited to any particular programming language or operating system.

It should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention can be practiced without these specific details. In other instances, well-known methods, structures, and techniques have not been shown in detail in order not to obscure an understanding of this description.

Thus, while there has been described what are believed to be the preferred embodiments of the invention, those skilled in the art will recognize that other and further modifications can be made thereto without departing from the spirit of the invention, and it is intended to claim all such changes and modifications as falling within the scope of the invention. For example, any formulas given above are merely representative of procedures that can be used. Functionality can be added or deleted from the block diagrams and operations are interchangeable among functional blocks. Steps can be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure further relates to the following aspects.

Example 1. A computer-implemented method including: deriving, by one or more processors, a model linking one or more environments with each of a plurality of conditions associated with the one or more environments; receiving, by the one or more processors, a resource associated with a condition from the plurality of conditions requested by a user; receiving, by the one or more processors, environment context data of the user; applying, by the one or more processors, the model to the environment context data of the user and the condition to identify a link of at least one environment, determined from the environment context data of the user, to the condition; based on the identified link, determining, by the one or more processors, a transferability of the resource requested; and based on the determining, changing, by the one or more processors, a status of the resource requested, wherein the changing of the status initiates a transfer evaluation of the resource requested.

Example 2. The computer-implemented method of example 1, wherein the model is a mapping table, and deriving the model includes: generating the mapping table based on known data for a plurality of resources requested by a plurality of users, the known data for each of the plurality of resources including: one or more of the plurality of conditions associated with the respective resource, environment context data of the respective user, and a transfer outcome of the respective resource.

Example 3. The computer-implemented method of example 2, further including: receiving, by the one or more processors, a transfer outcome of the resource as feedback from the transfer evaluation; and updating, by the one or more processors, the mapping table based on the feedback.

Example 4. The computer-implemented method of example 1, wherein the model is a machine learning model trained to learn the linking of the one or more environments with each of the plurality of conditions associated with the one or more environments based on a plurality of training data sets associated with a plurality of users, each training data set of the plurality of training data sets including: a known resource associated with one or more of the plurality of conditions requested by a respective user, a known environment context data of the respective user, and a known transfer outcome of the known resource.

Example 5. The computer-implemented method of example 4, further including: receiving, by the one or more processors, a transfer outcome of the resource as feedback from the transfer evaluation, wherein the machine learning model is updated based on the feedback.

Example 6. The computer-implemented method of any of examples 1-5, wherein the condition is indicated by a code included in a request for the resource, and the method further including: querying, by the one or more processors and using the code, the model or a separate data store configured to store a plurality of codes for each of the plurality of conditions associated with the one or more environments, to determine the resource is associated with the condition.

Example 7. The computer-implemented method of example 6, further including: receiving, by the one or more processors, transfer outcome data for transfer evaluated resources as feedback; and updating, by the one or more processors, the model or the separate data store based on the feedback to eliminate one or more codes from the plurality of codes.

Example 8. The computer-implemented method of any of examples 1-7, wherein receiving the environment context data of the user includes: generating an API call to request the environment context data of the user to one or more external resources; and receiving the environment context data of the user from the one or more external resources responsive to the API call.

Example 9. The computer-implemented method of any of examples 1-8, wherein determining the at least one environment from the environment context data of the user includes: identifying one or more entities from the environment context data of the user; and determining an association between the one or more entities and the at least one environment.

Example 10. The computer-implemented method of any of examples 1-9, further including: generating a summary including the condition, the at least one environment, and the identified link.

Example 11. The computer-implemented method of example 10, wherein the initiation of the transfer evaluation includes: automatically opening and assigning a case associated with the resource requested, wherein the summary is stored in association with the case.

Example 12. A system including: one or more processors; and at least one memory storing instructions that, when executed by the one or more processors, cause the one or more processors to perform operations including: deriving a model linking one or more environments with each of a plurality of conditions associated with the one or more environments; receiving a resource associated with a condition from the plurality of conditions requested by a user; receiving environment context data of the user; applying the model to the environment context data of the user and the condition to identify a link of at least one environment, determined from the environment context data of the user, to the condition; based on the identified link, determining a transferability of the resource requested; and based on the determining, changing a status of the resource requested, wherein the changing of the status initiates a transfer evaluation of the resource requested.

Example 13. The system of example 12, wherein the model is a mapping table, and deriving the model includes: generating the mapping table based on known data for a plurality of resources requested by a plurality of users, the known data for each of the plurality of resources including: one or more of the plurality of conditions associated with the respective resource, environment context data of the respective user, and a transfer outcome of the respective resource.

Example 14. The system of example 13, further including: receiving a transfer outcome of the resource as feedback from the transfer evaluation; and updating the mapping table based on the feedback.

Example 15. The system of example 12, wherein the model is a machine learning model trained to learn the linking of the one or more environments with each of the plurality of conditions associated with the one or more environments based on a plurality of training data sets associated with a plurality of users, each training data set of the plurality of training data sets including: a known resource associated with one or more of the plurality of conditions requested by a respective user, a known environment context data of the respective user, and a known transfer outcome of the known resource.

Example 16. The system of example 15, the techniques described herein relate to a system, further including: receiving a transfer outcome of the resource as feedback from the transfer evaluation, wherein the machine learning model is updated based on the feedback.

Example 17. The system of any of examples 12-16, wherein the condition is indicated by a code included in a request for the resource, and the operations further including: querying, using the code, the model or a separate data store configured to store a plurality of codes for each of the plurality of conditions associated with the one or more environments, to determine the resource is associated with the condition.

Example 18. The system of any of examples 12-17, wherein receiving the environment context data of the user includes: generating an API call to request the environment context data of the user to one or more external resources; and receiving the environment context data of the user from the one or more external resources responsive to the API call.

Example 19. The system of any of examples 12-18, wherein determining the at least one environment from the environment context data of the user includes: identifying one or more entities from the environment context data of the user; and determining an association between the one or more entities and the at least one environment.

Example 20. A non-transitory computer readable medium storing instructions which, when executed by one or more processors, cause the one or more processors to perform operations including: deriving a model linking one or more environments with each of a plurality of conditions associated with the one or more environments; receiving a resource associated with a condition from the plurality of conditions requested by a user; receiving environment context data of the user; applying the model to the environment context data of the user and the condition to identify a link of at least one environment, determined from the environment context data of the user, to the condition; based on the identified link, determining a transferability of the resource requested; and based on the determining, changing a status of the resource requested, wherein the changing of the status initiates a transfer evaluation of the resource requested.

Example 21. The computer-implemented method of example 4, wherein the training of the machine learning model is performed by the one or more processors.

Example 22. The computer-implemented method of example 4, wherein: the one or more processors are included in a first computing entity; and the training of the machine learning model is performed by one or more processors included in a second computing entity.

What is claimed is:

1. A computer-implemented method comprising:
deriving, by one or more processors, a model linking one or more environments with each of a plurality of conditions associated with the one or more environments;
receiving, by the one or more processors, a resource associated with a condition from the plurality of conditions requested by a user;
receiving, by the one or more processors, environment context data of the user;
applying, by the one or more processors, the model to the environment context data of the user and the condition to identify a link of at least one environment, determined from the environment context data of the user, to the condition;
based on the identified link, determining, by the one or more processors, a transferability of the resource requested; and
based on the determining, changing, by the one or more processors, a status of the resource requested, wherein the changing of the status initiates a transfer evaluation of the resource requested.

2. The computer-implemented method of claim 1, wherein the model is a mapping table, and deriving the model comprises:
generating the mapping table based on known data for a plurality of resources requested by a plurality of users, the known data for each of the plurality of resources including: one or more of the plurality of conditions associated with the respective resource, environment context data of the respective user, and a transfer outcome of the respective resource.

3. The computer-implemented method of claim 2, further comprising:
receiving, by the one or more processors, a transfer outcome of the resource as feedback from the transfer evaluation; and
updating, by the one or more processors, the mapping table based on the feedback.

4. The computer-implemented method of claim 1, wherein the model is a machine learning model trained to learn the linking of the one or more environments with each of the plurality of conditions associated with the one or more environments based on a plurality of training data sets associated with a plurality of users, each training data set of the plurality of training data sets including: a known resource associated with one or more of the plurality of conditions requested by a respective user, a known environment context data of the respective user, and a known transfer outcome of the known resource.

5. The computer-implemented method of claim 4, further comprising:
receiving, by the one or more processors, a transfer outcome of the resource as feedback from the transfer evaluation, wherein the machine learning model is updated based on the feedback.

6. The computer-implemented method of claim 1, wherein the condition is indicated by a code included in a request for the resource, and the method further comprising:
querying, by the one or more processors and using the code, the model or a separate data store configured to store a plurality of codes for each of the plurality of conditions associated with the one or more environments, to determine the resource is associated with the condition.

7. The computer-implemented method of claim 6, further comprising:
receiving, by the one or more processors, transfer outcome data for transfer evaluated resources as feedback; and
updating, by the one or more processors, the model or the separate data store based on the feedback to eliminate one or more codes from the plurality of codes.

8. The computer-implemented method of claim 1, wherein receiving the environment context data of the user comprises:
generating an API call to request the environment context data of the user to one or more external resources; and
receiving the environment context data of the user from the one or more external resources responsive to the API call.

9. The computer-implemented method of claim 1, wherein determining the at least one environment from the environment context data of the user comprises:
identifying one or more entities from the environment context data of the user; and
determining an association between the one or more entities and the at least one environment.

10. The computer-implemented method of claim 1, further comprising:
generating a summary including the condition, the at least one environment, and the identified link.

11. The computer-implemented method of claim 10, wherein the initiation of the transfer evaluation comprises:
automatically opening and assigning a case associated with the resource requested, wherein the summary is stored in association with the case.

12. A system comprising:
one or more processors; and
at least one memory storing instructions that, when executed by the one or more processors, cause the one or more processors to perform operations including:
deriving a model linking one or more environments with each of a plurality of conditions associated with the one or more environments;
receiving a resource associated with a condition from the plurality of conditions requested by a user;
receiving environment context data of the user;
applying the model to the environment context data of the user and the condition to identify a link of at least one environment, determined from the environment context data of the user, to the condition;
based on the identified link, determining a transferability of the resource requested; and
based on the determining, changing a status of the resource requested, wherein the changing of the status initiates a transfer evaluation of the resource requested.

13. The system of claim 12, wherein the model is a mapping table, and deriving the model comprises:
generating the mapping table based on known data for a plurality of resources requested by a plurality of users, the known data for each of the plurality of resources including: one or more of the plurality of conditions associated with the respective resource, environment context data of the respective user, and a transfer outcome of the respective resource.

14. The system of claim 13, further comprising:
receiving a transfer outcome of the resource as feedback from the transfer evaluation; and
updating the mapping table based on the feedback.

15. The system of claim 12, wherein the model is a machine learning model trained to learn the linking of the one or more environments with each of the plurality of conditions associated with the one or more environments based on a plurality of training data sets associated with a plurality of users, each training data set of the plurality of training data sets including: a known resource associated with one or more of the plurality of conditions requested by a respective user, a known environment context data of the respective user, and a known transfer outcome of the known resource.

16. The system of claim 15, further comprising:
receiving a transfer outcome of the resource as feedback from the transfer evaluation, wherein the machine learning model is updated based on the feedback.

17. The system of claim 12, wherein the condition is indicated by a code included in a request for the resource, and the operations further including:
querying, using the code, the model or a separate data store configured to store a plurality of codes for each of the plurality of conditions associated with the one or more environments, to determine the resource is associated with the condition.

18. The system of claim 12, wherein receiving the environment context data of the user comprises:
generating an API call to request the environment context data of the user to one or more external resources; and
receiving the environment context data of the user from the one or more external resources responsive to the API call.

19. The system of claim 12, wherein determining the at least one environment from the environment context data of the user comprises:
identifying one or more entities from the environment context data of the user; and
determining an association between the one or more entities and the at least one environment.

20. A non-transitory computer readable medium storing instructions which, when executed by one or more processors, cause the one or more processors to perform operations comprising:
deriving a model linking one or more environments with each of a plurality of conditions associated with the one or more environments;
receiving a resource associated with a condition from the plurality of conditions requested by a user;
receiving environment context data of the user;
applying the model to the environment context data of the user and the condition to identify a link of at least one environment, determined from the environment context data of the user, to the condition;
based on the identified link, determining a transferability of the resource requested; and
based on the determining, changing a status of the resource requested, wherein the changing of the status initiates a transfer evaluation of the resource requested.
